(19) United States
(12) Patent Application Publication
(10) Pub. No.: US 2026/0060904 A1
(43) Pub. Date: Mar. 5, 2026

(54) HIGH SPF SUNSCREEN CONTAINING HYDROGENATED CASTOR OIL

(71) Applicant: Kenvue Brands LLC, Summit, NJ (US)

(72) Inventors: Connor F. DEVOE, Morristown, NJ (US); Tatiana LUTS, Bridgewater, NJ (US)

(21) Appl. No.: 19/312,773

(22) Filed: Aug. 28, 2025

Related U.S. Application Data
(60) Provisional application No. 63/690,081, filed on Sep. 3, 2024.

(51) Int. Cl. A61K 8/06; A61K 8/35; A61K 8/37; A61K 8/40; A61K 8/81; A61K 8/92; A61Q 17/04

(52) U.S. Cl. CPC A61K 8/062; A61K 8/35; A61K 8/37; A61K 8/40; A61K 8/922; A61Q 17/04; A61K 8/8182; A61K 2800/26; A61K 2800/30

(57) ABSTRACT

The present invention provides a sunscreen composition comprising: (i) avobenzone; (ii) homosalate; (iii) octocrylene; (iv) octisalate; (v) hydrogenated castor oil; and (vi) carnauba wax; wherein the composition is a liquid, oil-in-water emulsion and provides an SPF of at least 70.

This application claims the benefit of U.S. provisional application 63/690,081 filed on Sep. 3, 2024, the complete disclosure of which is hereby incorporated herein by reference for all purposes.

The present invention provides a sunscreen composition comprising: (i) avobenzone; (ii) homosalate; (iii) octocrylene; (iv) octisalate; (v) hydrogenated castor oil; and (vi) carnauba wax; wherein the composition is a liquid, oil-in-water emulsion and provides an SPF of at least 70.

### BACKGROUND OF THE INVENTION

It is well known that prolonged exposure to ultraviolet radiation, such as from the sun, can lead to the formation of light dermatoses and erythemas, and increase the risk of skin cancers, such as melanoma. Exposure to UV radiation also accelerates skin aging, such as loss of skin elasticity and wrinkling.

For these reasons, sunscreen compositions are commonly used to provide protection from the sun, and a variety of sunscreen compositions are commercially available. Sunscreen compositions typically contain a combination of organic UV absorbers such as avobenzone, homosalate, octocrylene, and octisalate, and may be in the form of oil-in-water emulsions to provide good aesthetic properties. It is uncommon, however, for such compositions to provide very high SPF levels, such as 70 or 100 or greater even when using the maximum approved amounts of UV absorbers.

Applicants have now discovered very high SPF, oil-in-water emulsions comprising avobenzone, homosalate, octocrylene, and octisalate that also contain hydrogenated castor oil; and carnauba wax. These compositions are liquid, oil-in-water emulsions that advantageously provide in vivo or in vitro SPF levels of at least about 70, preferably at least about 75, more preferably at least about 100. As liquid emulsions, they also have good aesthetics as lotions.

### SUMMARY OF THE INVENTION

The present invention provides a sunscreen composition comprising: (i) avobenzone; (ii) homosalate; (iii) octocrylene; (iv) octisalate; (v) hydrogenated castor oil; and (vi) carnauba wax; wherein the composition is a liquid, oil-in-water emulsion and provides an in vivo SPF as measured by the IN VIVO SPF TEST METHOD of at least 70.

The invention also provides a sunscreen composition comprising: (i) about 3 weight % of avobenzone; (ii) about 15 weight % of homosalate; (iii) about 10 weight % of octocrylene; (iv) about 5 weight % of octisalate; (v) about 2 weight % of hydrogenated castor oil; (vi) about 2 weight % of carnauba wax; and (vii) about 1.5 weight % of triacontanyl PVP; wherein the composition is an oil-in-water emulsion having a shear viscosity of about 30,000 to about 1,400,000 cP at 25° C. that provides an in vivo SPF as measured by the IN VIVO SPF TEST METHOD of at least 70.

### DETAILED DESCRIPTION OF THE INVENTION

Unless defined otherwise, all technical and scientific terms used herein have the same meaning as commonly understood by one of ordinary skill in the art to which the invention belongs. All publications, patent applications, patents, and other references mentioned herein are incorporated by reference.

As used herein, “topically applying” means directly laying on or spreading on outer skin or the scalp, e.g., by use of the hands or an applicator such as a wipe, roller, or spray.

As used herein, “cosmetic” refers to a beautifying substance or preparation which preserves, restores, bestows, simulates, or enhances the appearance of bodily beauty or appears to enhance the beauty or youthfulness, specifically as it relates to the appearance of tissue or skin.

As used herein, “sunscreen composition” means a formulation that is a lotion that absorbs and/or reflects some of the sun's ultraviolet (UV) radiation and thus helps protect against negative effects of sun exposure, e.g., sunburn, premature aging, etc.

As used herein, “cosmetically effective amount” means an amount of a physiologically active compound or composition sufficient for treating one or more conditions, but low enough to avoid serious side effects. The cosmetically effective amount of the compound or composition will vary with the condition being treated, the age and physical condition of the end user, the severity of the condition being treated/prevented, the duration of the treatment, the nature of other treatments, the specific compound or product/composition employed, the cosmetically acceptable carrier utilized, and like factors.

As used herein, “cosmetically acceptable” means that the ingredients the term describes are suitable for use in contact with tissues (e.g., the skin) without undue toxicity, incompatibility, instability, irritation, allergic response, or the like.

As used herein, a “cosmetically acceptable active agent” is a compound (synthetic or natural) that has a cosmetic or therapeutic effect on the skin.

As used herein, “treatment or treating” refers to mitigating, reducing, preventing, improving, or eliminating the presence or signs of a condition or disorder.

As used herein, “substantially free of” means the ingredient referred to is not directly or intentionally added. Preferably, “substantially free of” means containing less than about 1% by weight. More preferably “substantially free of” means containing less than about 0.5% by weight. Even more preferably “substantially free of” means containing less than about 0.1% by weight. The present sunscreen composition may be completely free of an ingredient, i.e., contain none of the ingredient.

As used herein, “UV absorber” means an active ingredient whose specific function is to absorb radiation in some portion of the ultraviolet spectrum (290 nm-400 nm), such as one having an extinction coefficient of at least about 1000 mol-1 cm−1, for at least one wavelength within the above-defined ultraviolet spectrum. UV absorbers include for example approved sun filters and are distinguished from compounds having other functions that also happen to absorb UV radiation.

As used herein, “UV blocker” means an active ingredient whose specific function is to reflect or scatter UV radiation, for example mineral sunscreens such as titanium dioxide or zinc oxide.

As used herein, “phase-stable” means the water and oil phases of an emulsion do not appreciably separate at room temperature for at least two weeks.

Unless otherwise indicated, a percentage or concentration refers to a percentage or concentration by weight (i.e., % (W/W) based on the total weight of the composition. Unless stated otherwise, all ranges are inclusive of the endpoints, e.g., “from 4 to 9” includes the endpoints 4 and 9.

### UV Absorbers

The sunscreen composition of the invention comprises a combination of avobenzone, homosalate, octocrylene, and octisalate as UV absorbers.

Preferably, the composition is substantially free, or completely free, of oxybenzone.

The composition comprises about 2 to about 10% by weight of avobenzone based on the total weight of the composition. Preferably, the composition comprises about 3% by weight of avobenzone based on the total weight of the composition.

The composition comprises about 5 to about 15% by weight of homosalate based on the total weight of the composition. Preferably, the composition comprises about 15% by weight of homosalate based on the total weight of the composition.

The composition comprises about 5 to about 12% by weight of octocrylene based on the total weight of the composition. Preferably, the composition comprises about 10% by weight of octocrylene based on the total weight of the composition.

The composition comprises about 2 to about 10% by weight of octisalate based on the total weight of the composition. Preferably, the composition comprises about 5% by weight of octisalate based on the total weight of the composition.

In one embodiment, the composition comprises about 3% by weight avobenzone, about 15% by weight homosalate, about 10% by weight octocrylene, and about 5% by weight octisalate.

Avobenzone is commercially available, for example under the trade name Neo Heliopan 357 from Symrise.

Homosalate is commercially available, for example under the trade name Neo Heliopan HMS from Symrise.

Octocrylene is commercially available, for example under the trade name Neo Heliopan 303 from Symrise.

Octisalate is commercially available, for example under the trade name Neo Heliopan OS/BP from Symrise.

The composition need not contain additional UV absorbers or UV blockers to obtain a high SPF. Accordingly, it is preferably free of them. However, it may optionally contain other UV absorbers or UV blockers known in the art.

Examples of UV absorbers include, but are not limited to: methoxycinnamate derivatives such as octyl methoxycinnamate and isoamyl methoxycinnamate; camphor derivatives such as 4-methyl benzylidene camphor, camphor benzalkonium methosulfate, and terephthalylidene dicamphor sulfonic acid; other salicylate derivatives; benzoic acid derivatives such as aminobenzoic acid and octyldimethyl para-amino benzoic acid; other β,β-diphenylacrylates; dioctyl butamido triazone; octyl triazone; menthyl anthranilate; triazone derivatives such as ethylhexyl triazone (Uvinul® T150); diethylhexyl butamido triazone (UVASorb® HEB); bis-ethylhexyloxyphenol methoxyphenyl triazine (Tinosorb® S), benzoate derivatives such as diethylamino hydroxybenzoyl hexyl benzoate (Uvinul® A Plus), benzotriazole derivatives such as drometrizole trisiloxane (Mexoryl® XL), methylene bis-benzotriazolyl tetramethylbutylphenol (Tinosorb® M); tris-biphenyl triazine; (2-{4-[2-(4-Diethylamino-2-hydroxy-benzoyl)-benzoyl]-piperazine-1-carbonyl}-phenyl)-(4-diethylamino-2-hydroxy-phenyl)-methanone; phenylbenzimidazole sulfonic acid (Eusolex® 232); merocyanine derivatives; bis(butylbenzoate) diaminotriazine aminopropylsiloxane; and bis-ethylhexyloxyphenol methoxyphenyl triazine encapsulated in a polymer matrix.

Similarly, the composition need not contain polymeric organic UV absorbers. As used herein, “polymeric organic UV absorber” means a polymeric compound comprising organic chromophores attached to a polymer chain. For instance, the polymer chain may be a polysiloxane having for example an average molecular weight of greater than 6000 Daltons. Examples of such polysiloxane UV absorbers include, without limitation, Parsol® SLX commercially available from DSM and polysilicone-15. These polysiloxanes absorb in the UVB (λmax=312 nm) part of the spectrum and are typically combined with UVA filters to achieve broad-spectrum protection.

However, optionally the composition may contain one or more polymeric organic UV absorbers. Because of their relatively high molecular weight, they are regarded as substantially non-absorbing to the skin.

### Hydrogenated Castor Oil

The composition comprises hydrogenated castor oil (Castor Wax or Hydrogenated Castor Wax). The hydrogenated castor oil may be fully or partially hydrogenated (saturated). Preferably the hydrogenated castor oil is fully hydrogenated. Non-hydrogenated castor oil (commonly referred to as “castor oil”) is not suitable for the present invention. In addition, while variations of hydrogenated castor oil exist, such as such as “PEG-40 Hydrogenated Castor Oil” or polymers containing hydrogenated castor oil such “hydrogenated castor oil/sebacic acid copolymer,” the present invention utilizes hydrogenated castor oil rather than such variations.

The composition typically comprises about 0.5 to about 5% by weight of hydrogenated castor oil, preferably about 1 to about 3% by weight of hydrogenated castor oil, based on the total weight of the composition.

Hydrogenated castor oil is commercially available, for example from BASF under the trade name Cutina HR Flakes.

### Carnauba Wax

The composition comprises carnauba wax (Copernicia Cerifera (Carnauba) Wax).

The composition typically comprises about 0.5 to about 5% by weight of carnauba wax, preferably about 1 to about 3% by weight of carnauba wax, based on the total weight of the composition.

Carnauba wax is commercially available, for example from Strahl and Pitsch Inc. under the trade name Carnauba Wax #1 Flakes N.F. SP-63.

The weight ratio of hydrogenated castor oil to carnauba wax in the composition may range from about 0.10 to about 10.

### Film Former

The composition may optionally comprise the film former tricontanyl PVP (tricontanyl povidone or 2-Pyrrolidinone, 1-ethenyl-, polymer with 1-triacontene).

The composition may comprise about 0.5 to about 4% by weight of triacontanyl PVP, preferably about 1 to about 3% by weight of triacontanyl PVP, based on the total weight of the composition.

Triacontanyl PVP is commercially available, for example from ISP Freetown Fine Chemicals Inc. under the trade name Antaron™ WP-660 polymer.

### SPF

The composition provides Sun Protection Factor (SPF) of at least about 70, preferably at least about 75, more preferably at least about 100. SPF may be measured in vivo or in vitro.

The SPF of the composition may be tested in vivo using the methodology set forth in the US FDA OTC monograph for sunscreens (U.S. Food and Drug Administration Over-the-Counter Monograph M020: Sunscreen Drug Products for Over-the-Counter Human Use (Posted Sep. 24, 2021); Final Administrative Order (OTC000006), effective upon enactment of the Coronavirus Aid, Relief, and Economic Security Act (CARES Act), Public Law 116-136, on Mar. 27, 2020) (“IN VIVO SPF TEST METHOD”).

In one embodiment, the composition has an SPF as measured by the IN VIVO SPF TEST METHOD of at least about 70. In another embodiment, the composition has an SPF as measured by the IN VIVO SPF TEST METHOD of at least about 75. In another embodiment, the composition has an SPF as measured by the IN VIVO SPF TEST METHOD of at least about 100.

Alternatively, the SPF of the composition may be tested using the following IN VITRO SPF TEST METHOD. The baseline transmission of a PMMA plate (substrate, available from Helioscience, Marseille, France) is measured for UV absorbance using calibrated Labsphere® UV-1000S UV transmission analyzer or a Labsphere® UV-2000S UV transmission analyzer (Labsphere, North Sutton, N.H., USA). A test sample is then applied to the PMMA plate using an application density of about 0.75 mg/cm2 by rubbing into a uniform thin layer with the operator's finger. The sample is allowed to dry for 15 minutes and then measured for UV absorbance in the same way. The absorbance measures are used to calculate SPF as known in the art using the following equation:

$$SPF in vitro = \frac{{\int }_{λ = 290 nm}^{λ = 400 nm} E ( λ ) * I ( λ ) * d λ}{{\int }_{λ = 290 nm}^{λ = 400 nm} E ( λ ) * I ( λ ) * {10}^{{A}_{0} ( λ )} * d λ}$$

in which:
E(λ)=Erythema action spectrum;
I(λ)=Spectral irradiance received from the UV source;
A₀(λ)=Mean monochromatic absorbance of the test product layer before UV exposure; and
d(λ)=Wavelength step (1 nm).

In one embodiment, the composition has an SPF as measured by the IN VITRO SPF TEST METHOD of at least about 70. In another embodiment, the composition has an SPF as measured by the IN VITRO SPF TEST METHOD of at least about 75. In another embodiment, the composition has an SPF as measured by the IN VITRO SPF TEST METHOD of at least about 100.

### Shear Viscosity

The composition is advantageously a lotion rather than a solid or stick in spite of containing carnauba wax. Accordingly, it is a liquid, having a shear viscosity preferably of less than about 2,000,000 cP at 25° C. It may for example have a shear viscosity of about 30,000 to about 1,400,000 cP at 25° C. As a liquid, lotion, it has the consumer preferred aesthetics of appealing appearance, consistency, ease of application, and look and feel on the skin. Shear viscosity may be measured according to methods well known in the art using a Brookfield Viscometer. Multiple spindle sizes and rotational speeds may be used to measure the viscosity, including spindles T-C and T-D and rotational speeds ranging from 0.5 rpm to 5 rpm.

### Topical Composition

The sunscreen composition can be used by topically applying to a mammal, e.g., by the direct laying on, wiping or spreading of the composition on the skin, hair, or nails of a mammal, particularly a human.

The sunscreen composition is in the form of an oil-in-water emulsion containing a continuous water phase and a discontinuous oil phase dispersed within the continuous water phase. The composition is preferably phase stable.

The oil phase may be such that it is present in discrete droplets or units having an average diameter of about 0.5 micron to about 1000 microns, such as from about 1 micron to about 100 microns.

The percentage by weight of water phase included in the compositions may range from about 35 to about 60% by weight of the composition, such as from about 40% to about 50% by weight of the composition.

The sunscreen composition may be prepared using mixing and blending methodology well known in the sunscreen and cosmetic art. Typically, the sunscreen composition is produced by preparing an oil phase containing the oil soluble and oil-miscible ingredients, including the UV absorbers, hydrogenated castor oil; and carnauba wax and triacontanyl PVP (if present); and preparing a water phase by mixing water with the water-soluble or water-miscible ingredients. The oil phase and the water phase may then be mixed in a manner sufficient to disperse the oil phase substantially homogeneously in the water phase such that the water phase is continuous and the oil phase discontinuous.

The composition may be combined with a “cosmetically acceptable topical carrier,” i.e., a carrier for topical use that capable of containing the other ingredients dispersed or dissolved therein and possessing acceptable properties rendering it safe to use topically.

The composition may optionally comprise a wide variety of additional oil-soluble materials and/or water-soluble materials conventionally used in compositions for use on skin, at their art-established levels. For example, surfactants, pearlescent or opacifying agents, thickeners, emollients, conditioners, humectants, chelating agents, exfoliants, oil absorbers, and additives that enhance the appearance, feel, or fragrance of the cleansing composition, such as colorants, fragrances, preservatives, pH adjusting agents, and the like, can be included, provided they do not interfere with the high SPF.

In one embodiment the composition is substantially free or, or free of, fatty alcohols. Particular examples of fatty alcohols include behenyl alcohol, arachidyl alcohol, stearyl alcohol and cetyl alcohol.

Water soluble or water dispersible polymers may be added to the compositions. The water dispersible polymers are comprised of a water-insoluble polymer that is typically micronized and dispersed into a water carrier, possibly with the use of a surface active dispersing aid. The water dispersible polymers are capable of forming a film and improving water resistance of the compositions. Examples of water-soluble polymers include Polyaldo® 10-1-L (Polyglyceryl-10 Laurate), available from Lonza. Examples of water dispersible polymers include water dispersible polyurethanes, such as Baycusan® C1000 (Polyurethane-34), available from Bayer, Dow Corning®2501 (Bis-PEG-18 Methyl Ether Dimethyl Silane), available from Dow Corning, Eastman AQ™ 38S (Polyester-5), available from Eastman Chemical, and Intelimer® 8600 (C8-22 Alkyl Acrylates/Methacrylic Acid Crosspolymer) available from Air Products.

The sunscreen composition may also comprise certain additional film formers, provided they do not interfere with the SPF of the composition. Acceptable additional film formers include polyethylene, which is available from New Phase Technologies as Performalene® 400, a polyethylene having a molecular weight of 400. Another suitable film former is polyethylene 2000 (molecular weight of 2000), which is available from New Phase Technologies as Performalene®. Another suitable film former is synthetic wax, also available from New Phase Technologies as Performa® V-825. Other typical film-formers include VP/hexadecene copolymer, acrylates/acrylamide copolymer, acrylates copolymer, acrylates/C12-C22 alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica campestris/Aleuritis fordi Oil copolymer, decamethyl cyclopentasiloxane (and) trimethylsiloxysilicate, Dimethicone; Acrylates/Dimethicone Copolymer and mixtures thereof. In some cases, the film former is acrylates/C12-C22 alkylmethacrylate copolymer sold under the tradename Allianz™ OPT by Ashland.

The composition may comprise one or more oil absorbents such as aluminum starch octenylsuccinate or silica. The composition may comprise a combination of aluminum starch octenylsuccinate or silica.

Suitable emollients for use in the composition include mineral oils, petrolatum, vegetable oils (e.g. triglycerides such as caprylic/capric triglyceride, Helianthus Annuus (Sunflower) Seed Oil), waxes and other mixtures of fatty esters, including but not limited to esters of glycerol (e.g, isopropyl palmitate, isopropyl myristate, diisopropyl adipate, dicaprylyl carbonate, C12-15 alkyl benzoate, isononyl isononanoate, neopentyl glycol diheptonoate, neopentyl glycol dibenzoate, butyloctyl salicylate, ethylhexylmethoxycrylene), hydrocarbons (e.g, isododecane, isohexadecane, squalane), ethers (e.g, PPG-15 stearyl ether, dicaprylyl ether), silicone oils such as dimethicone, and jojoba esters. In certain embodiments, mixtures of triglycerides (e.g. caprylic/capric triclycerides) and esters of glycols (e.g. isopropyl myristate).

The sunscreen composition may comprise one or more emulsifiers. In one embodiment, the sunscreen composition comprises one or more of PEG-100 stearate, glyceryl stearate, and sodium acryloyldimethyltaurate/VP Crosspolymer. In another embodiment, the sunscreen composition comprises one or more of PEG-100 stearate, glyceryl stearate, and potassium cetyl phosphate. Preferably, the composition contains a combination of all three compounds. In a third embodiment, the sunscreen composition comprises one or more of PEG-100 stearate, glyceryl stearate, and acrylates C10/30 alkyl acrylate crosspolymer.

Examples of suitable solvents include propylene glycol, polyethylene glycol, polypropylene glycol, glycerol, 1,2,4-butanetriol, sorbitol esters, 1,2,6-hexanetriol, ethanol, and mixtures thereof.

In certain embodiments, the composition includes a pigment suitable for providing color or hiding power. The pigment may be one suitable for use in a color cosmetic product, including compositions for application to the hair, nails and/or skin, especially the face. Color cosmetic compositions include, but are not limited to, foundations, concealers, primers, blush, mascara, eyeshadow, eyeliner, lipstick, nail polish and tinted moisturizers. The pigment suitable for providing color or hiding power may be composed of iron oxides, including red and yellow iron oxides, titanium dioxide, ultramarine and chromium or chromium hydroxide colors, and mixtures thereof. The pigment may be a lake pigment, e.g. an organic dye such as azo, indigoid, triphenylmethane, anthraquinone, and xanthine dyes that are designated as D&C and FD&C blues, browns, greens, oranges, reds, yellows, etc., precipitated onto inert binders such as insoluble salts. Examples of lake pigments include Red #6, Red #7, Yellow #5, Violet #2 and Blue #1. The pigment may be an interference pigment. Examples of interference pigments include those containing mica substrates, bismuth oxycloride substrates, and silica substrates, for instance mica/bismuth oxychloride/iron oxide pigments commercially available as CHROMALITE pigments (BASF), titanium dioxide and/or iron oxides coated onto mica such as commercially available FLAMENCO pigments (BASF), mica/titanium dioxide/iron oxide pigments including commercially available KTZ pigments (Kobo products), CELLINI pearl pigments (BASF), and borosilicate-containing pigments such as REFLECKS pigments (BASF).

In one embodiment, the sunscreen composition comprises a humectant. Examples of humectants include, without limitation to, glycerin, pentylene glycol, butylene glycol.

The composition may comprise one or more SPF boosters, for example styrene/acrylates copolymer, butyloctyl salicylate, or ethylhexyl methoxycrylene. The composition may comprise both styrene/acrylates copolymer and ethylhexyl methoxycrylene.

In another embodiment, the sunscreen composition has a pH of about 5.0 to about 7.0, for example about 6.0.

The composition may further comprise one or more other cosmetically acceptable active agents include for example anti-acne agents, shine control agents, anti-microbial agents, anti-inflammatory agents, anti-mycotic agents, anti-parasite agents, external analgesics, antioxidants, keratolytic agents, moisturizers, nutrients, vitamins, energy enhancers, anti-perspiration agents, astringents, deodorants, firming agents, anti-callous agents, and agents for skin conditioning.

The amount of other cosmetically active agents may range from about 0.001% to about 20% by weight of the composition, e.g., about 0.005% to about 10% by weight of the composition, such as about 0.01% to about 5% by weight of the composition.

The cosmetically acceptable active agent may be selected for instance from D-panthenol carotenoids, ceramides, polyunsaturated fatty acids, essential fatty acids, enzymes such as laccase, enzyme inhibitors, minerals, steroids such as hydrocortisone, 2-dimethylaminoethanol, copper salts such as copper chloride, peptides like argireline, syn-ake, acetyl dipeptide-31 amide and those containing copper, coenzyme Q10, amino acids such as proline, vitamins, lactobionic acid, acetyl-coenzyme A, niacin, riboflavin, thiamin, ribose, electron transporters such as NADH and FADH2, natural extracts such as from aloe vera, feverfew (Chrysanthemum Parthenium (Feverfew) Flower/Leaf/Steam Juice), oatmeal, dill, blackberry, princess tree, Picia anomala, and chicory, resorcinols such as 4-hexyl resorcinol, curcuminoids, sugar amines such as N-acetyl glucosamines, and derivatives and mixtures thereof.

Examples of vitamins and their derivatives include, but are not limited to, vitamin A, vitamin B's such as vitamin B3, niacinamide, vitamin B5, and vitamin B112, vitamin C, vitamin K, and different forms of vitamin E such as alpha, beta, gamma or delta tocopherols or their mixtures, and derivatives thereof.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, propolis and Zingiber Officinale (Ginger) Root Extract.

In one embodiment, the composition comprises one or both of tocopherol and feverfew extract (for example Chrysanthemum Parthenium (Feverfew) Flower/Leaf/Stem Juice).

In one embodiment, the composition comprises phenoxyethanol, chlorphenesin, ethylhexylglycerin and caprylyl glycol. This specific combination of preservatives surprisingly increases the shelf life of compositions according to the invention. Preferably, the composition comprises about 0.7% by weight phenoxyethanol, about 0.25% by weight chlorphenesin, about 0.5% by weight ethylhexylglycerin and about 0.6% by weight caprylyl glycol.

The following non-limiting examples further illustrate the invention.

### Example 1

Compositions 1-5 according to the invention were made using the ingredients shown in Table 1. The compositions were liquid, oil-in-water emulsions. The compositions were all prepared generally by mixing an oil phase and a water phase in a manner sufficient to disperse the oil phase substantially homogeneously in the water phase, such that the water phase was continuous and the oil phase was discontinuous. The oil phase ingredients included acrylates/C10-30 alkyl acrylate crosspolymer, avobenzone, caprylyl methicone, cetyl dimethicone, copernicia cerifera (carnauba) wax, chlorphenesin, dimethicone, dimethicone/acrylates copolymer, ethylhexyl methoxycerylene, glyceryl stearate, homosalate, hydrogenated castor oil, hydrolyzed jojoba esters, jojoba esters, octisalate, octocrylene, PEG-100 stearate, styrene/acrylates copolymer, tocopheryl acetate, and triacontanyl PVP. The water phase ingredients included aluminum starch octenylsuccinate, caprylyl glycol, chrysanthemum parthenium (feverfew) flower/leaf/stem juice, disodium EDTA, ethylhexylglycerin, ethylhexyl stearate, hydroxyacetophenone, phenoxyethanol, potassium cetyl phosphate, silica, sodium acryloyldimethyltaurate/VP crosspolymer, sodium hydroxide, sodium polyacrylate, trideceth-6, water and xanthan gum.

Compositions 1, 2, 4, and 5 were tested for in vivo SPF using the IN VIVO SPF TEST METHOD.

In addition, Compositions 3 and 4 were evaluated in a consumer test for the following aesthetic attributes: Appearance, Consistency, Ease of Application, Feel of the Skin with Sunscreen on, Look of the Skin with Sunscreen On, and Overall Smell. The evaluation was performed as follows. Consumers were given samples of the formula compositions included in the test and were advised to use the samples at least 3 times per week while performing outdoor activities for a period of 2 weeks. After using the samples for the 2 weeks, the test participants rated on a scale from 1 to 9 how much they liked each formula based on the aesthetic attributes listed above. Answers in the top third of the range (7-9) were assessed as a positive rating and the percentages listed in Table 1 are the percentage of participants agreeing for that specific attribute.

The results are shown in Table 1. Compositions 1-5 were phase stable and Compositions 1, 2, 4, and 5 had excellent, high in vivo SPF levels, all greater than 100. In addition, Compositions 3 and 4 had excellent aesthetic attributes, particularly with respect to Consistency and Ease of Application. These results demonstrate that a surprising combination of very high SPF and excellent aesthetics are achievable in a liquid, lotion form with a sunscreen composition comprising: (i) avobenzone; (ii) homosalate; (iii) octocrylene; (iv) octisalate; (v) hydrogenated castor oil; and (vi) carnauba wax.

TABLE 1

| | | Percentage (% w/w) | | | | |
|---|---|---|---|---|---|---|
| | | Composi- | Composi- | Composi- | Composi- | Composi- |
| US INCI Name | Function | tion 1 | tion 2 | tion 3 | tion 4 | tion 5 |
| Homosalate | Sun Filter | 15.00 | 15.00 | 15.00 | 15.00 | 15.00 |
| Octocrylene | Sun Filter | 10.00 | 10.00 | 10.00 | 10.00 | 10.00 |
| Octisalate | Sun Filter | 5.00 | 5.00 | 5.00 | 5.00 | 5.00 |
| Avobenzone | Sun Filter | 3.00 | 3.00 | 3.00 | 3.00 | 3.00 |
| Water | Solvent | 43.55 | 43.35 | 43.25 | 45.10 | 45.68 |
| Styrene/Acrylates | Film Former | 3.00 | 3.00 | 3.00 | 3.00 | 3.00 |
| Copolymer | | | | | | |
| Ethylhexyl | Skin | 2.80 | 3.00 | 3.00 | 3.00 | 3.00 |
| Methoxycrylene | Conditioner | | | | | |
| Copernicia Cerifera | Oil | 2.00 | 2.00 | 2.00 | 1.50 | 2.00 |
| (Carnauba) Wax | Thickener | | | | | |
| Hydrogenated Castor Oil | Oil | 2.00 | 2.00 | 2.00 | 1.00 | 2.00 |
| | Thickener | | | | | |
| Silica | Absorbent | 2.00 | 2.00 | 2.00 | 2.00 | 2.00 |
| PEG-100 Stearate; | Emulsifier | 1.70 | 1.70 | 1.70 | 2.00 | 1.70 |
| Glyceryl Stearate | | | | | | |
| Triacontanyl PVP | Film Former | 1.50 | 1.50 | 1.50 | 3.00 | 1.50 |
| Potassium Cetyl | Emulsifier | 1.40 | 1.40 | 1.40 | — | — |
| Phosphate | | | | | | |
| Caprylyl Methicone | Slip Modifier | 1.00 | 1.00 | 1.00 | 0.50 | 1.00 |
| Aluminum Starch | Absorbent | 1.00 | 1.00 | 1.00 | 1.00 | 1.00 |
| Octenylsuccinate | | | | | | |
| Dimethicone; | Film Former | 1.00 | 1.00 | 1.00 | 1.00 | 1.00 |
| Acrylates/Dimethicone | | | | | | |
| Copolymer | | | | | | |
| Cetyl Dimethicone | Slip Modifier | 0.75 | 0.75 | 0.75 | 0.75 | 0.75 |
| Phenoxyethanol | Preservative | 0.70 | 0.70 | 0.70 | 0.70 | 0.70 |
| Caprylyl Glycol | Skin | 0.60 | 0.60 | 0.60 | 0.60 | — |
| | Conditioner | | | | | |
| Ethylhexylglycerin | Skin | 0.50 | 0.50 | 0.50 | 0.50 | — |
| | Conditioner | | | | | |
| Sodium Polyacrylate; | Thickener | 0.40 | 0.40 | 0.40 | — | — |
| Ethylhexyl Stearate; | | | | | | |
| Trideceth-6 | | | | | | |
| Xanthan Gum | Thickener | 0.30 | 0.30 | 0.30 | — | — |
| Chlorphenesin | Preservative | 0.25 | 0.25 | 0.25 | 0.25 | 0.27 |
| Hydrolyzed Jojoba | Emollient | 0.25 | 0.25 | 0.25 | 0.25 | 0.25 |
| Esters; Jojoba Esters; | | | | | | |
| Water | | | | | | |
| Disodium EDTA | Chelating | 0.20 | 0.20 | 0.20 | 0.20 | 0.20 |
| | Agent | | | | | |
| Tocopheryl Acetate | Antioxidant | 0.10 | 0.10 | 0.10 | 0.10 | 0.10 |
| Chrysanthemum | Antioxidant | — | — | 0.10 | 0.10 | 0.10 |
| Parthenium (Feverfew) | | | | | | |
| Flower/Leaf/Stem Juice | | | | | | |
| Sodium | Emulsifier | — | — | — | 0.45 | — |
| Acryloyldimethyltaurate/ | | | | | | |
| VP Crosspolymer | | | | | | |
| Acrylates/C10-30 Alkyl | Emulsifier | — | — | — | — | 0.20 |
| Acrylate Crosspolymer | | | | | | |
| Hydroxyacetophenone | Skin | — | — | — | — | 0.50 |
| | Conditioner | | | | | |
| Sodium Hydroxide | pH Adjustor | — | — | — | — | 0.05 |
| Claimable In-Vivo SPF | — | 101 | 106 | — | 100 | 101 |
| Viscosity (cP)¹ | | 350,000- | 400,000- | 400,000- | 30,000- | 30,000- |
| | | 800,000 | 1,400,000 | 1,400,000 | 175,000 | 225,000 |
| Appearance | — | — | — | 89% | 85% | — |
| Consistency | — | — | — | 89% | 90% | — |
| Ease of Application | — | — | — | 92% | 90% | — |
| Feel of the Skin with the | — | — | — | 89% | 81% | — |
| sunscreen on | | | | | | |
| Look of the Skin with the | — | — | — | 89% | 83% | — |
| sunscreen on | | | | | | |
| Overall Smell | — | — | — | 77% | 69% | — |

¹Composition 1 viscosity tested using Spindles TC and TD and rotational speeds 1 RPM and 2.5 RPM;
Composition 2 and 3 viscosity tested using Spindle TC at 0.5 RPM;
Composition 4 viscosity tested using Spindle TC at 5 RPM;
Composition 5 viscosity tested using Spindle TC at 4 RPM.

### Example 2

A series of comparative compositions were made using the ingredients shown in Table 2. They were prepared in the manner described in Example 1. The comparative compositions were identical other than the oil thickener and amount of water (q.s.) used. Composition 6 contained no oil thickener. Compositions 7-12 contained one of the following oil thickeners: synthetic beeswax, behenyl alcohol, hydrogenated castor oil, beeswax, candelilla wax, or carnauba wax (Copernicia Cerifera (Carnauba) Wax). The compositions were tested for in vitro SPF using the IN VITRO SPF METHOD. Composition 6 was used as the reference for the SPF testing, and the percentage change in SPF from the reference was calculated for the other compositions.

The results are shown in Table 2. Compositions 9 and 12 containing either hydrogenated castor oil or carnauba wax demonstrated the highest SPF values.

TABLE 2

| | | Percentage (% w/w) | | | | | | |
|---|---|---|---|---|---|---|---|---|
| | | Composi- | Composi- | Composi- | Composi- | Composi- | Composi- | Composi- |
| US INCI Name | Function | tion 6 | tion 7 | tion 8 | tion 9 | tion 10 | tion 11 | tion 12 |
| Avobenzone | Sun Filter | 3.00 | 3.00 | 3.00 | 3.00 | 3.00 | 3.00 | 3.00 |
| Homosalate | Sun Filter | 15.00 | 15.00 | 15.00 | 15.00 | 15.00 | 15.00 | 15.00 |
| Octisalate | Sun Filter | 5.00 | 5.00 | 5.00 | 5.00 | 5.00 | 5.00 | 5.00 |
| Octocrylene | Sun Filter | 10.00 | 10.00 | 10.00 | 10.00 | 10.00 | 10.00 | 10.00 |
| Synthetic Beeswax | Oil Thickener | — | 2.00 | — | — | — | — | — |
| Behenyl Alcohol | Oil Thickener | — | — | 2.00 | — | — | — | — |
| Hydrogenated Castor | Oil Thickener | — | — | — | 2.00 | — | — | — |
| Oil | | | | | | | | |
| Beeswax | Oil Thickener | — | — | — | — | 2.00 | — | — |
| Candelilla Wax | Oil Thickener | — | — | — | — | — | 2.00 | — |
| Copernicia Cerifera | Oil Thickener | — | — | — | — | — | — | 2.00 |
| (Carnauba) Wax | | | | | | | | |
| Cetyl Dimethicone | Emollient | 0.75 | 0.75 | 0.75 | 0.75 | 0.75 | 0.75 | 0.75 |
| Caprylyly Methicone | Emollient | 1.00 | 1.00 | 1.00 | 1.00 | 1.00 | 1.00 | 1.00 |
| PEG-100 Stearate; | Emulsifier | 1.70 | 1.70 | 1.70 | 1.70 | 1.70 | 1.70 | 1.70 |
| Glyceryl Stearate | | | | | | | | |
| Potassium Cetyl | Emulsifier | 1.40 | 1.40 | 1.40 | 1.40 | 1.40 | 1.40 | 1.40 |
| Phosphate | | | | | | | | |
| Ethylhexylglycerin | Skin Conditioner | 0.50 | 0.50 | 0.50 | 0.50 | 0.50 | 0.50 | 0.50 |
| Xanthan Gum | Thickener | 0.30 | 0.30 | 0.30 | 0.30 | 0.30 | 0.30 | 0.30 |
| Disodium EDTA | Chelating Agent | 0.20 | 0.20 | 0.20 | 0.20 | 0.20 | 0.20 | 0.20 |
| Chlorphenesin | Preservative | 0.25 | 0.25 | 0.25 | 0.25 | 0.25 | 0.25 | 0.25 |
| Water | Solvent | 56.85 | 54.85 | 54.85 | 54.85 | 54.85 | 54.85 | 54.85 |
| Sodium Polyacrylate; | Thickener | 0.40 | 0.40 | 0.40 | 0.40 | 0.40 | 0.40 | 0.40 |
| Ethylhexyl Stearate; | | | | | | | | |
| Trideceth-6 | | | | | | | | |
| Caprylyl Glycol | Skin Conditioner | 0.60 | 0.60 | 0.60 | 0.60 | 0.60 | 0.60 | 0.60 |
| Dimethicone; | Film Former | 1.00 | 1.00 | 1.00 | 1.00 | 1.00 | 1.00 | 1.00 |
| Acrylates/Dimethicone | | | | | | | | |
| Copolymer | | | | | | | | |
| Phenoxyethanol | Preservative | 0.70 | 0.70 | 0.70 | 0.70 | 0.70 | 0.70 | 0.70 |
| Hydrolyzed Jojoba | Emollient | 0.25 | 0.25 | 0.25 | 0.25 | 0.25 | 0.25 | 0.25 |
| Esters; Jojoba Esters; | | | | | | | | |
| Water | | | | | | | | |
| Aluminum Starch | Absorbent | 1.00 | 1.00 | 1.00 | 1.00 | 1.00 | 1.00 | 1.00 |
| Octenylsuccinate | | | | | | | | |
| Tocopheryl Acetate | Antioxidant | 0.10 | 0.10 | 0.10 | 0.10 | 0.10 | 0.10 | 0.10 |
| Percent Change of in- | — | — | 55% | −17% | 154% | 34% | 135% | 170% |
| vitro SPF from | | | | | | | | |
| Reference Formula | | | | | | | | |
| (Formula 6) | | | | | | | | |

### Example 3

Composition 9 was evaluated with three additional compositions containing 3 weight % avobenzone, 15 weight % homosalate, 10 weight % octocrylene, and 5 weight % octisalate. All the compositions contained (i) hydrogenated castor oil, (ii) carnauba wax, or (iii) both, but were otherwise the same (water q.s.) and prepared in the manner described in Example 1. The ingredients are shown in Table 3. Composition 15 was made according to the invention containing 2 weight % hydrogenated castor oil and 2 weight % carnauba wax.

The compositions were tested for in vitro SPF using the IN VITRO SPF METHOD. Composition 15 served as the reference formula for in vitro SPF testing. The compositions were also tested for in vivo SPF using the IN VIVO SPF TEST METHOD.

The results are shown in Table 3. Composition 15 containing both hydrogenated castor oil and carnauba wax according to the invention had the highest in vitro and in vivo SPF results. It was surprisingly and advantageously the most efficacious composition of the group.

TABLE 3

| | | Percentage (% w/w) | | | |
|---|---|---|---|---|---|
| | | Composition | Composition | Composition | Composition |
| US INCI Name | Function | 9 | 13 | 14 | 15 |
| Avobenzone | Sun Filter | 3.00 | 3.00 | 3.00 | 3.00 |
| Homosalate | Sun Filter | 15.00 | 15.00 | 15.00 | 15.00 |
| Octisalate | Sun Filter | 5.00 | 5.00 | 5.00 | 5.00 |
| Octocrylene | Sun Filter | 10.00 | 10.00 | 10.00 | 10.00 |
| Hydrogenated Castor | Oil | 2.00 | 4.00 | — | 2.00 |
| Oil | Thickener | | | | |
| Copernicia Cerifera | Oil | — | — | 4.00 | 2.00 |
| (Carnauba) Wax | Thickener | | | | |
| Cetyl Dimethicone | Emollient | 0.75 | 0.75 | 0.75 | 0.75 |
| Caprylyly Methicone | Emollient | 1.00 | 1.00 | 1.00 | 1.00 |
| PEG-100 Stearate; | Emulsifier | 1.70 | 1.70 | 1.70 | 1.70 |
| Glyceryl Stearate | | | | | |
| Potassium Cetyl | Emulsifier | 1.40 | 1.40 | 1.40 | 1.40 |
| Phosphate | | | | | |
| Ethylhexylglycerin | Skin | 0.50 | 0.50 | 0.50 | 0.50 |
| | Conditioner | | | | |
| Xanthan Gum | Thickener | 0.30 | 0.30 | 0.30 | 0.30 |
| Disodium EDTA | Chelating | 0.20 | 0.20 | 0.20 | 0.20 |
| | Agent | | | | |
| Chlorphenesin | Preservative | 0.25 | 0.25 | 0.25 | 0.25 |
| Water | Solvent | 54.85 | 52.85 | 52.85 | 52.85 |
| Sodium Polyacrylate; | Thickener | 0.40 | 0.40 | 0.40 | 0.40 |
| Ethylhexyl Stearate; | | | | | |
| Trideceth-6 | | | | | |
| Caprylyl Glycol | Skin | 0.60 | 0.60 | 0.60 | 0.60 |
| | Conditioner | | | | |
| Dimethicone; | Film Former | 1.00 | 1.00 | 1.00 | 1.00 |
| Acrylates/Dimethicone | | | | | |
| Copolymer | | | | | |
| Phenoxyethanol | Preservative | 0.70 | 0.70 | 0.70 | 0.70 |
| Hydrolyzed Jojoba | Emollient | 0.25 | 0.25 | 0.25 | 0.25 |
| Esters; Jojoba Esters; | | | | | |
| Water | | | | | |
| Aluminum Starch | Absorbent | 1.00 | 1.00 | 1.00 | 1.00 |
| Octenylsuccinate | | | | | |
| Tocopheryl Acetate | Antioxidant | 0.10 | 0.10 | 0.10 | 0.10 |
| Percent change of in- | — | −34% | −21% | −41% | — |
| vitro SPF from | | | | | |
| Reference Formula | | | | | |
| (Formula 15) | | | | | |
| Mean In-vivo SPF | — | — | 73.3 ± 8.9 | — | 84.9 ± 18.2 |

### Example 4

Composition 15 was evaluated with three additional compositions containing 3 weight % avobenzone, 15 weight % homosalate, 10 weight % octocrylene, and 5 weight % octisalate. The additional compositions contained hydrogenated castor oil and a film former selected from triacontanyl PVP, VP/eicosene copolymer, and VP/hexadecene copolymer. Composition 15, as described above, was made according to the invention containing hydrogenated castor oil and carnauba wax. The compositions were otherwise the same (water q.s.) and prepared as described in Example 1. The ingredients are shown in Table 4.

The compositions were tested for in vitro SPF using the IN VITRO SPF METHOD. Composition 15 served as the reference formula.

The results are shown in Table 4.

TABLE 4

| | | Percentage (% w/w) | | | |
|---|---|---|---|---|---|
| | | Composition | Composition | Composition | Composition |
| US INCI Name | Function | 15 | 16 | 17 | 18 |
| Avobenzone | Sun Filter | 3.00 | 3.00 | 3.00 | 3.00 |
| Homosalate | Sun Filter | 15.00 | 15.00 | 15.00 | 15.00 |
| Octisalate | Sun Filter | 5.00 | 5.00 | 5.00 | 5.00 |
| Octocrylene | Sun Filter | 10.00 | 10.00 | 10.00 | 10.00 |
| Hydrogenated Castor | Oil | 2.00 | 2.00 | 2.00 | 2.00 |
| Oil | Thickener | | | | |
| Copernicia Cerifera | Oil | 2.00 | — | — | — |
| (Carnauba) Wax | Thickener | | | | |
| Triacontanyl PVP | Film Former | — | 1.50 | — | — |
| VP/Eicosene | Film Former | — | — | 1.50 | — |
| Copolymer | | | | | |
| VP/Hexadecene | Film Former | — | — | — | 1.50 |
| Copolymer | | | | | |
| Cetyl Dimethicone | Emollient | 0.75 | 0.75 | 0.75 | 0.75 |
| Caprylyly Methicone | Emollient | 1.00 | 1.00 | 1.00 | 1.00 |
| PEG-100 Stearate; | Emulsifier | 1.70 | 1.70 | 1.70 | 1.70 |
| Glyceryl Stearate | | | | | |
| Potassium Cetyl | Emulsifier | 1.40 | 1.40 | 1.40 | 1.40 |
| Phosphate | | | | | |
| Ethylhexylglycerin | Skin | 0.50 | 0.50 | 0.50 | 0.50 |
| | Conditioner | | | | |
| Xanthan Gum | Thickener | 0.30 | 0.30 | 0.30 | 0.30 |
| Disodium EDTA | Chelating | 0.20 | 0.20 | 0.20 | 0.20 |
| | Agent | | | | |
| Chlorphenesin | Preservative | 0.25 | 0.25 | 0.25 | 0.25 |
| Water | Solvent | 52.85 | 53.35 | 53.35 | 53.35 |
| Sodium Polyacrylate; | Thickener | 0.40 | 0.40 | 0.40 | 0.40 |
| Ethylhexyl Stearate; | | | | | |
| Trideceth-6 | | | | | |
| Caprylyl Glycol | Skin | 0.60 | 0.60 | 0.60 | 0.60 |
| | Conditioner | | | | |
| Dimethicone; | Film Former | 1.00 | 1.00 | 1.00 | 1.00 |
| Acrylates/Dimethicone | | | | | |
| Copolymer | | | | | |
| Phenoxyethanol | Preservative | 0.70 | 0.70 | 0.70 | 0.70 |
| Hydrolyzed Jojoba | Emollient | 0.25 | 0.25 | 0.25 | 0.25 |
| Esters; Jojoba Esters; | | | | | |
| Water | | | | | |
| Aluminum Starch | Absorbent | 1.00 | 1.00 | 1.00 | 1.00 |
| Octenylsuccinate | | | | | |
| Tocopheryl Acetate | Antioxidant | 0.10 | 0.10 | 0.10 | 0.10 |
| Percent change of in- | — | — | 28% | 16% | −4% |
| vitro SPF from | | | | | |
| Reference Formula | | | | | |
| (Formula 15) | | | | | |

1. A sunscreen composition comprising:
(i) avobenzone;
(ii) homosalate;
(iii) octocrylene;
(iv) octisalate;
(v) hydrogenated castor oil; and
(vi) carnauba wax;
wherein the composition is a liquid, oil-in-water emulsion and provides an in vivo SPF as measured by the IN VIVO SPF TEST METHOD of at least 70.

2. The sunscreen composition of claim 1 further comprising triacontanyl PVP.

3. The sunscreen composition of claim 1 substantially free of fatty alcohols.

4. The sunscreen composition of claim 1 substantially free of oxybenzone.

5. The sunscreen composition of claim 1 having a shear viscosity of less than 2,000,000 cP at 25° C.

6. A sunscreen composition comprising:
(i) about 3 weight % of avobenzone;
(ii) about 15 weight % of homosalate;
(iii) about 10 weight % of octocrylene;
(iv) about 5 weight % of octisalate;
(v) about 2 weight % of hydrogenated castor oil;
(vi) about 2 weight % of carnauba wax; and
(vii) about 1.5 weight % of triacontanyl PVP;
wherein the composition is a liquid, oil-in-water emulsion having a shear viscosity of about 30,000 to about 1,400,000 cP at 25° C. that provides an in vivo SPF as measured by the IN VIVO SPF TEST METHOD of at least 70.
